(19) United States
(12) Patent Application Publication
(10) Pub. No.: US 2007/0257603 A1
(43) Pub. Date: Nov. 8, 2007

(54) FLUORENE COMPOUND AND ORGANIC LIGHT-EMITTING DEVICE USING THE COMPOUND

(75) Inventors: Koichi Suzuki, Yokohama-shi (JP); Kazunori Ueno, Ebina-shi (JP); Hiroshi Tanabe, Yokohama-shi (JP); Satoru Shiobara, Tokyo (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/668,414

(22) Filed: Jan. 29, 2007

(30) Foreign Application Priority Data
Feb. 2, 2006 (JP) 2006/025208

(51) Int. Cl. H01J 1/62; C07C 25/13; C07C 19/08; C07C 255/52

(52) U.S. Cl. 313/504; 546/285; 548/528; 558/420; 570/127

(57) ABSTRACT

A novel fluorene compound is provided which has at least one fluorene group represented by the following general formula [III] or [IV]:

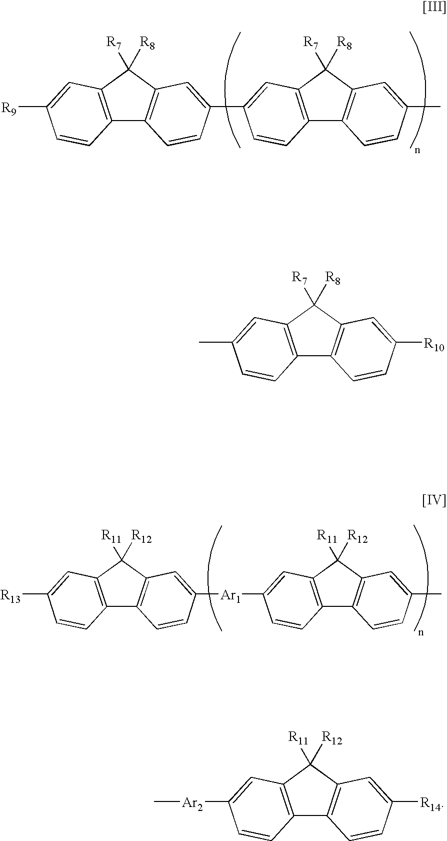

### BACKGROUND OF THE INVENTION

1. Field of the Invention

The present invention relates to a novel organic compound and an organic light-emitting device using the compound.

2. Description of the Related Art

An organic light-emitting device has a structure in which a thin film comprising a fluorescent organic compound or phosphorescent organic compound is interposed between an anode and a cathode. By injecting electrons and holes (positive holes) from the electrodes into the device, excitons of the fluorescent organic compound or phosphorescent organic compound are generated, and light radiated when the excitons return to a ground state is utilized.

Recently, research has been conducted on a light-emitting device made by a coating method using a polyfluorene-based soluble polymer (Japanese Patent Application Laid-Open No. 2000-100572).

However, at present, the light-emitting device disclosed in the above patent document is not sufficient in terms of the initial characteristics such as light emission efficiency and the durability characteristics such as resistance to degradation of luminance due to long-term light emission, so that further improvement of the characteristics is still required. In the polyfluorene-based soluble polymer, in order to improve the solubility in a solvent, long chain alkyl groups or bulky alkyl groups are contained as soluble substituents in the repeating units. However, such long chain alkyl groups or bulky alkyl groups facilitate entanglement in a molecule or entanglement of molecules to be apt to cause aggregation or the like in the state of a solution or a film of the polymer and also lower the compatibility with a guest material. Such factors have an influence on the light emission efficiency and the life of the device.

### SUMMARY OF THE INVENTION

It is, therefore, an object of the present invention to provide a novel fluorene compound.

It is another object of the present invention to provide an organic light-emitting device which has a high light emission efficiency and good durability.

It is still another object of the present invention to provide an organic light-emitting device which can easily be produced by a coating method which is relatively less expensive.

It is yet another object of the present invention to provide a novel fluorene compound and an organic light-emitting device using the compound.

The present inventors have found that a specified fluorene compound containing a trifluoromethyl group as a soluble substituent has a high solubility in a solvent, and that an organic light-emitting device which is produced using the compound for a light-emitting layer especially by employing a coating method has excellent initial characteristics and durability characteristics.

Such a compound can be used not only for a coating method in which a solution is applied in a planar direction such as spin coating later described in examples of the present invention but also for a linear drawing method in which a solution is disposed linearly at a predetermined position.

Here, the linear drawing method refers to a method in which a solution prepared by dissolving an organic compound in a solvent is disposed linearly at a predetermined position and which is so termed because of resembling the way of drawing a line with an ink held in a pen (or a nozzle).

(1) According to the present invention, there is provided a fluorene compound represented by the following general formula [III].

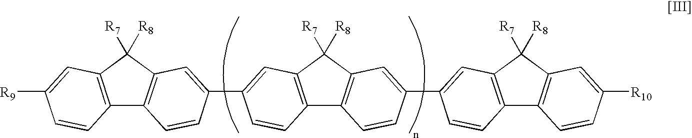

In the general formula [III], R₇ and R₈ each represent an alkyl group, a substituted or unsubstituted aralkyl group, a substituted or unsubstituted aryl group, or a trifluoromethyl group with the proviso that at least one of R₇'s and R₈'s is a trifluoromethyl group. R₇'s and R₈'s attached to different fluorene groups may be the same or different from each other. R₇'s and R₈'s attached to the same fluorene group may be the same or different from each other.

R₉ and R₁₀ each represent a hydrogen atom, a substituted or unsubstituted alkyl group, a substituted or unsubstituted aralkyl group, a substituted or unsubstituted aryl group, a substituted or unsubstituted heterocyclic group, a substituted or unsubstituted fused polycyclic aromatic group, a substituted or unsubstituted fused polycyclic heterocyclic group, a substituted or unsubstituted aryloxy group, a halogen atom, or a cyano group. R₉ and R₁₀ may be the same or different from each other. n represents an integer of 1 to 10.

(2) According to the present invention, there is also provided an organic light-emitting device comprising a pair of electrodes including an anode and a cathode, and an organic compound layer provided between the pair of electrodes, wherein the organic compound layer comprises the compound set forth in (1) above.

(3) According to the present invention, there is also provided the organic light-emitting device set forth in (2) above, wherein the organic compound layer is a light-emitting layer.

(4) According to the present invention, there is further provided a fluorene compound represented by the following general formula [IV].

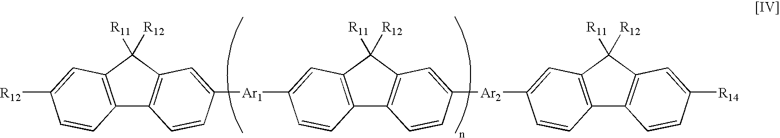

In the general formula [IV], R₁₁ and R₁₂ each represent an alkyl group, a substituted or unsubstituted aralkyl group, a substituted or unsubstituted aryl group, or a trifluoromethyl group with the proviso that at least one of R₁₁'s and R₁₂'s is a trifluoromethyl group. R₁₁'s and R₁₂'s attached to different fluorene groups may be the same or different from each other. R₁₁'s and R₁₂'s attached to the same fluorene group may be the same or different from each other.

R₁₃ and R₁₄ each represent a hydrogen atom, a substituted or unsubstituted alkyl group, a substituted or unsubstituted aralkyl group, a substituted or unsubstituted aryl group, a substituted or unsubstituted heterocyclic group, a substituted or unsubstituted fused polycyclic aromatic group, a substituted or unsubstituted fused polycyclic heterocyclic group, a substituted or unsubstituted aryloxy group, a halogen atom, or a cyano group. R₁₁ and R₁₂ may be the same or different from each other. Ar₁ and Ar₂ each represent a substituted or unsubstituted divalent aryl group or a substituted or unsubstituted divalent fused polycyclic aromatic group. Ar₁ and Ar₂ may be the same or different from each other. n represents an integer of 1 to 10.

(5) According to the present invention, there is also provided an organic light-emitting device comprising a pair of electrodes including an anode and a cathode, and an organic compound layer provided between the pair of electrodes, wherein the organic compound layer comprises the compound set forth in (4) above.

(6) According to the present invention, there is also provided the organic light-emitting device set forth in (5) above, wherein the organic compound layer is a light-emitting layer.

The organic light-emitting device using the fluorene compound according to the present invention provides light emission with a high luminance at a low applied voltage and is also excellent in durability.

Moreover, the device can be produced by use of a vacuum deposition or coating method, and a relatively low-cost, large-area device can easily be produced.

Further features of the present invention will become apparent from the following description of exemplary embodiments with reference to the attached drawings.

### BRIEF DESCRIPTION OF THE DRAWINGS

FIG. 1 is a schematic cross-sectional view showing an example of the organic light-emitting device in accordance with the present invention.

FIG. 2 is a schematic cross-sectional view showing another example of the organic light-emitting device in accordance with the present invention.

FIG. 3 is a schematic cross-sectional view showing still another example of the organic light-emitting device in accordance with the present invention.

FIG. 4 is a schematic cross-sectional view showing yet another example of the organic light-emitting device in accordance with the present invention.

FIG. 5 is a schematic cross-sectional view showing again another example of the organic light-emitting device in accordance with the present invention.

FIG. 6 is a schematic cross-sectional view showing yet still another example of the organic light-emitting device in accordance with the present invention.

FIG. 7 is a schematic cross-sectional view showing yet again another example of the organic light-emitting device in accordance with the present invention.

### DESCRIPTION OF THE EMBODIMENTS

The present invention will now be described in detail.

First, the fluorene compound according to the present invention will be described.

Here, the fluorene compound according to the present invention is represented by the following general formula [III] or [IV].

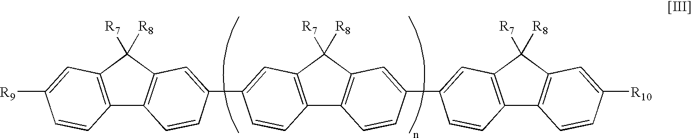

In the general formula [III], R₇ and R₈ each represent an alkyl group, a substituted or unsubstituted aralkyl group, a substituted or unsubstituted aryl group, or a trifluoromethyl group with the proviso that at least one of R₇'s and R₈'s is a trifluoromethyl group. R₇'s and R₈'s attached to different fluorene groups may be the same or different from each other. R₇'s and R₈'s attached to the same fluorene group may be the same or different from each other.

R₉ and R₁₀ each represent a hydrogen atom, a substituted or unsubstituted alkyl group, a substituted or unsubstituted aralkyl group, a substituted or unsubstituted aryl group, a substituted or unsubstituted heterocyclic group, a substituted or unsubstituted fused polycyclic aromatic group, a substituted or unsubstituted fused polycyclic heterocyclic group, a substituted or unsubstituted aryloxy group, a halogen atom, or a cyano group. R₉ and R₁₀ may be the same or different from each other. n represents an integer of 1 to 10.

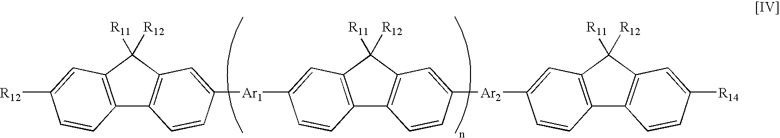

In the general formula [IV], R₁₁ and R₁₂ each represent an alkyl group, a substituted or unsubstituted aralkyl group, a substituted or unsubstituted aryl group, or a trifluoromethyl group with the proviso that at least one of R₁₁'s and R₁₂'s is a trifluoromethyl group. R₁₁ 's and R₁₂'s attached to different fluorene groups may be the same or different from each other. R₁₁'s and R₁₂'s attached to the same fluorene group may be the same or different from each other.

R₁₃ and R₁₄ each represent a hydrogen atom, a substituted or unsubstituted alkyl group, a substituted or unsubstituted aralkyl group, a substituted or unsubstituted aryl group, a substituted or unsubstituted heterocyclic group, a substituted or unsubstituted fused polycyclic aromatic group, a substituted or unsubstituted fused polycyclic heterocyclic group, a substituted or unsubstituted aryloxy group, a halogen atom, or a cyano group. R₁₁ and R₁₂ may be the same or different from each other. Ar₁ and Ar₂ each represent a substituted or unsubstituted divalent aryl group or a substituted or unsubstituted divalent fused polycyclic aromatic group. Ar₁ and Ar₂ may be the same or different from each other. n represents an integer of 1 to 10.

There are shown below specific examples of the substituents in the general formulae [III] and [IV], which are, however, given for the purpose of illustration and not by way of limitation.

As the alkyl group, there are included methyl group, ethyl group, and n-propyl group.

As the aralkyl group, there are included benzyl group and phenethyl group.

As the aryl group, there are included phenyl group, biphenyl group, and terphenyl group.

As the heterocyclic group, there are included thienyl group, pyrrolyl group, pyridyl group, bipyridyl group, oxazolyl group, oxadiazolyl group, thiazolyl group, and thiadiazolyl group.

As the fused polycyclic aromatic group, there are included naphthyl group and phenanthryl group.

As the fused polycyclic heterocyclic group, there are included quinolyl group, carbazolyl group, acridinyl group, and phenanthryl group.

As the aryloxy group, there are included phenoxyl group and naphthoxyl group.

As the substituted amino group, there are included dimethylamino group, diethylamino group, diphenylamino group, ditolylamino group, dianisolylamino group, fluorenylphenylamino group, difluorenylamino group, naphthylphenylamino group, and dinaphthylamino group.

As the halogen atom, there are included fluorine, chlorine, bromine, and iodine.

As the divalent aryl group, there are included phenylene group, biphenylene group, and terphenylene group.

As the divalent fused polycyclic aromatic group, there are included naphthylene group and anthrylene group.

As the substituents which the above described substituents may further posses, there are included alkyl groups such as methyl group, ethyl group, and propyl group; aralkyl groups such benzyl group and phenethyl group; aryl groups such as phenyl group, biphenyl group, and terphenyl group; heterocyclic groups such as thienyl group, pyrrolyl group, pyridyl group, bipyridyl group, oxazolyl group, oxadiazolyl group, thiazolyl group, and thiadiazolyl group; fused polycyclic aromatic groups such as naphthyl group and phenanthryl group; fused polycyclic heterocyclic groups such as quinolyl group, carbazolyl group, acridinyl group, and phenanthryl group; aryloxy groups such as phenoxyl group and naphthoxyl group; substituted amino groups such as dimethylamino group, diethylamino group, diphenylamino group, ditolylamino group, dianisolylamino group, fluorenylphenylamino group, difluorenylamino group, naphthylphenylamino group, and dinaphthylamino group; and halogen atoms such as fluorine, chlorine, bromine, and iodine.

Next, typical examples of fluorene compounds according to the present invention will be given below. However, it should be understood that the same is by way of illustration and example only and the invention is not limited thereto.

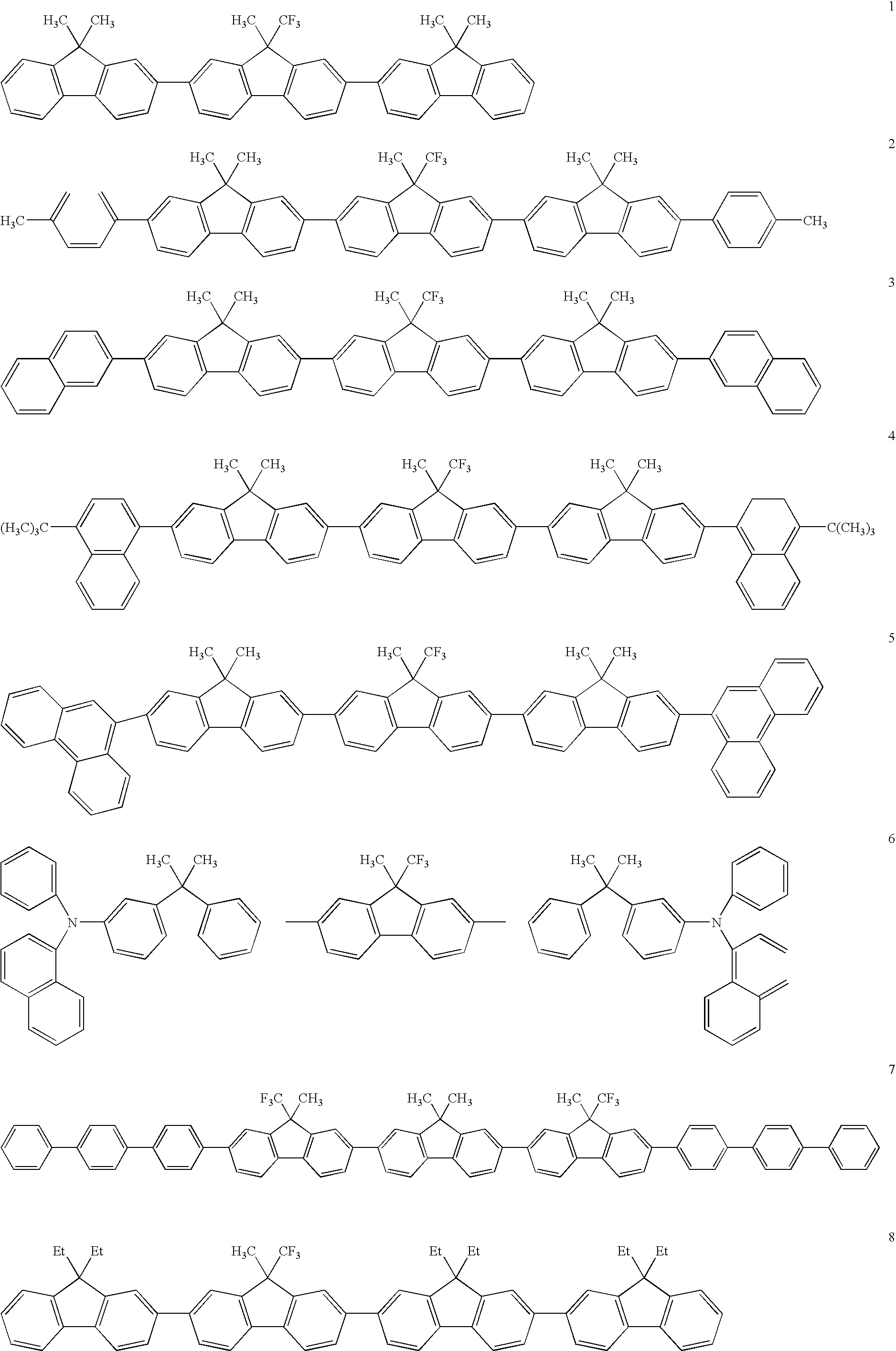

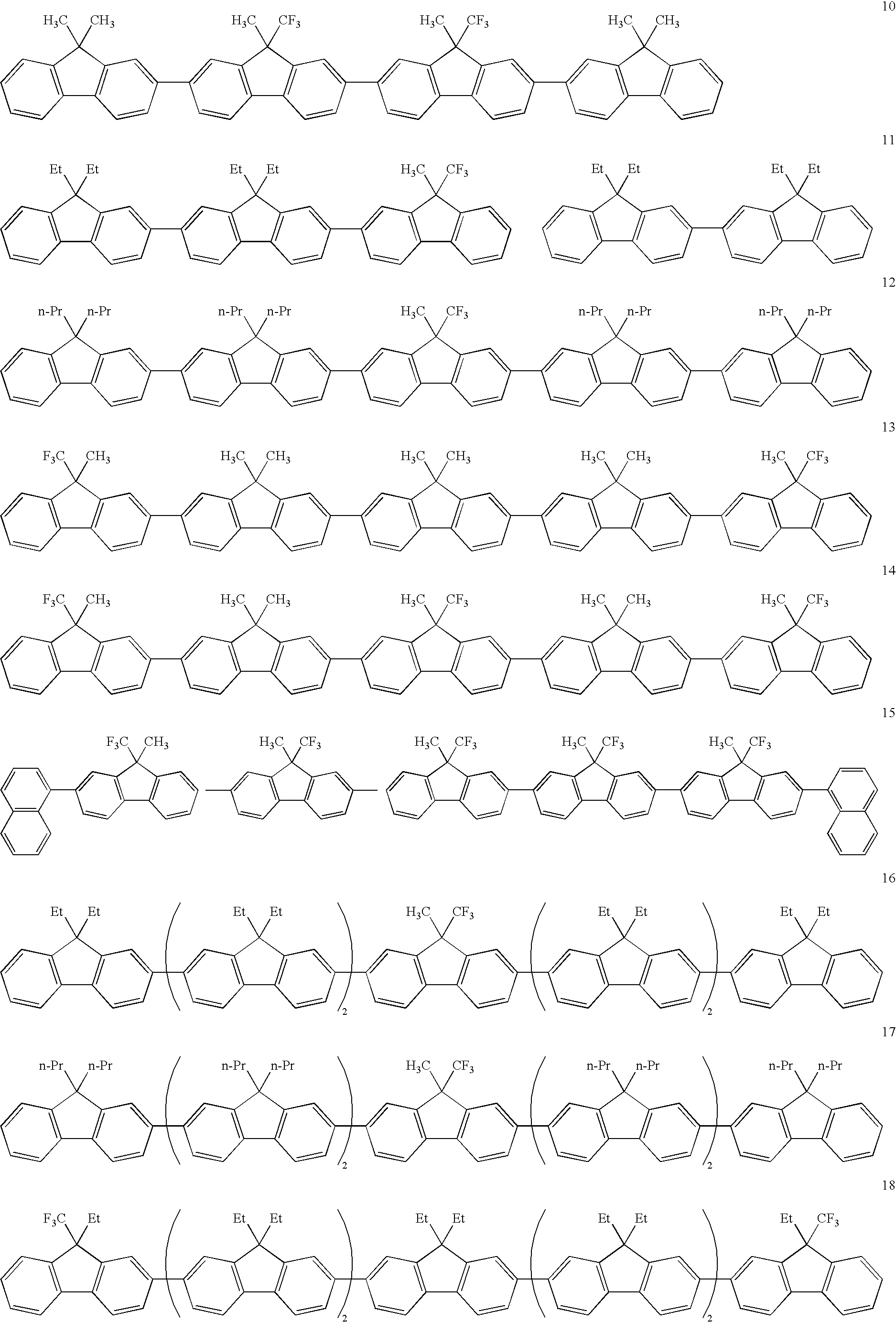

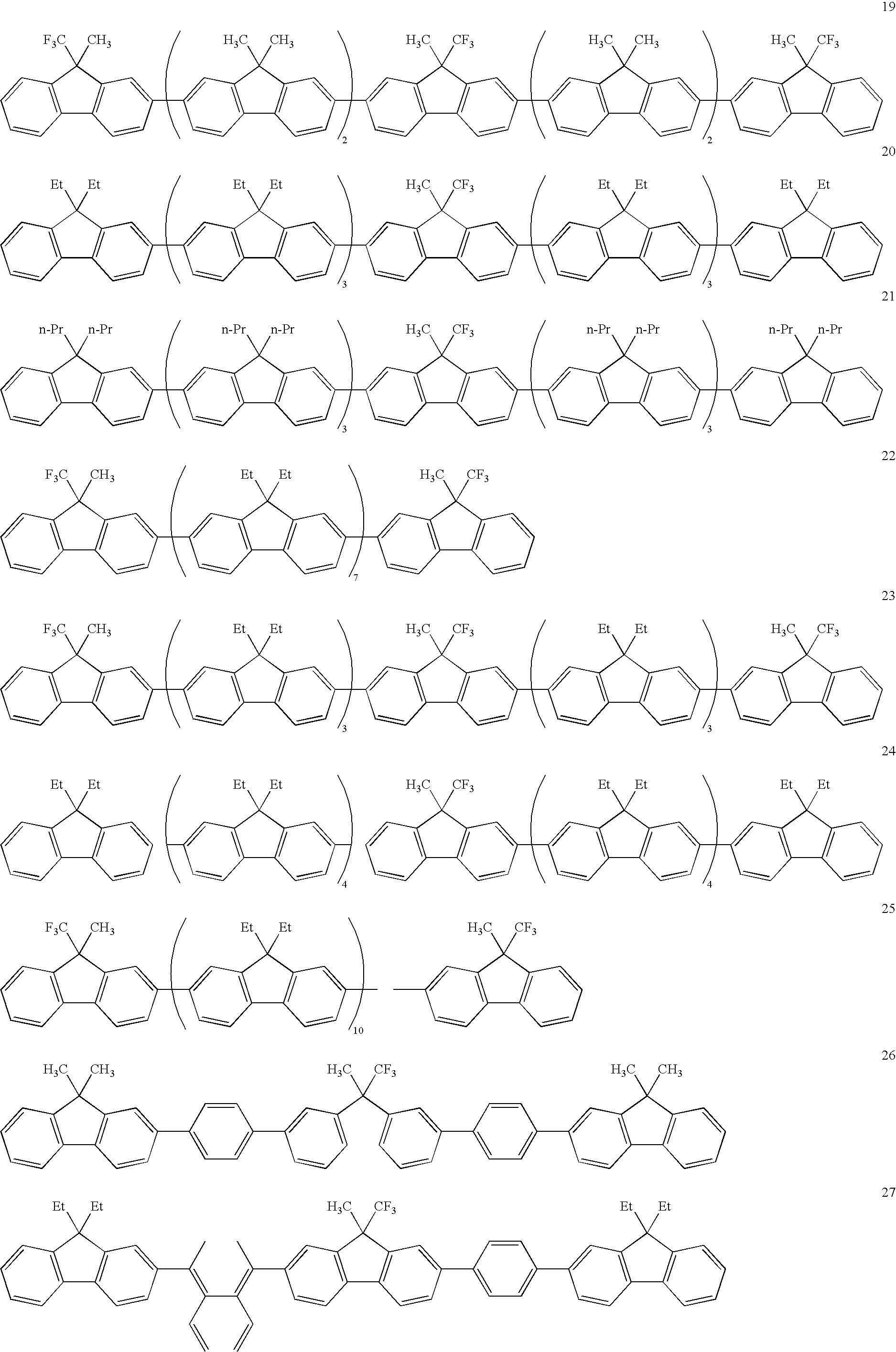

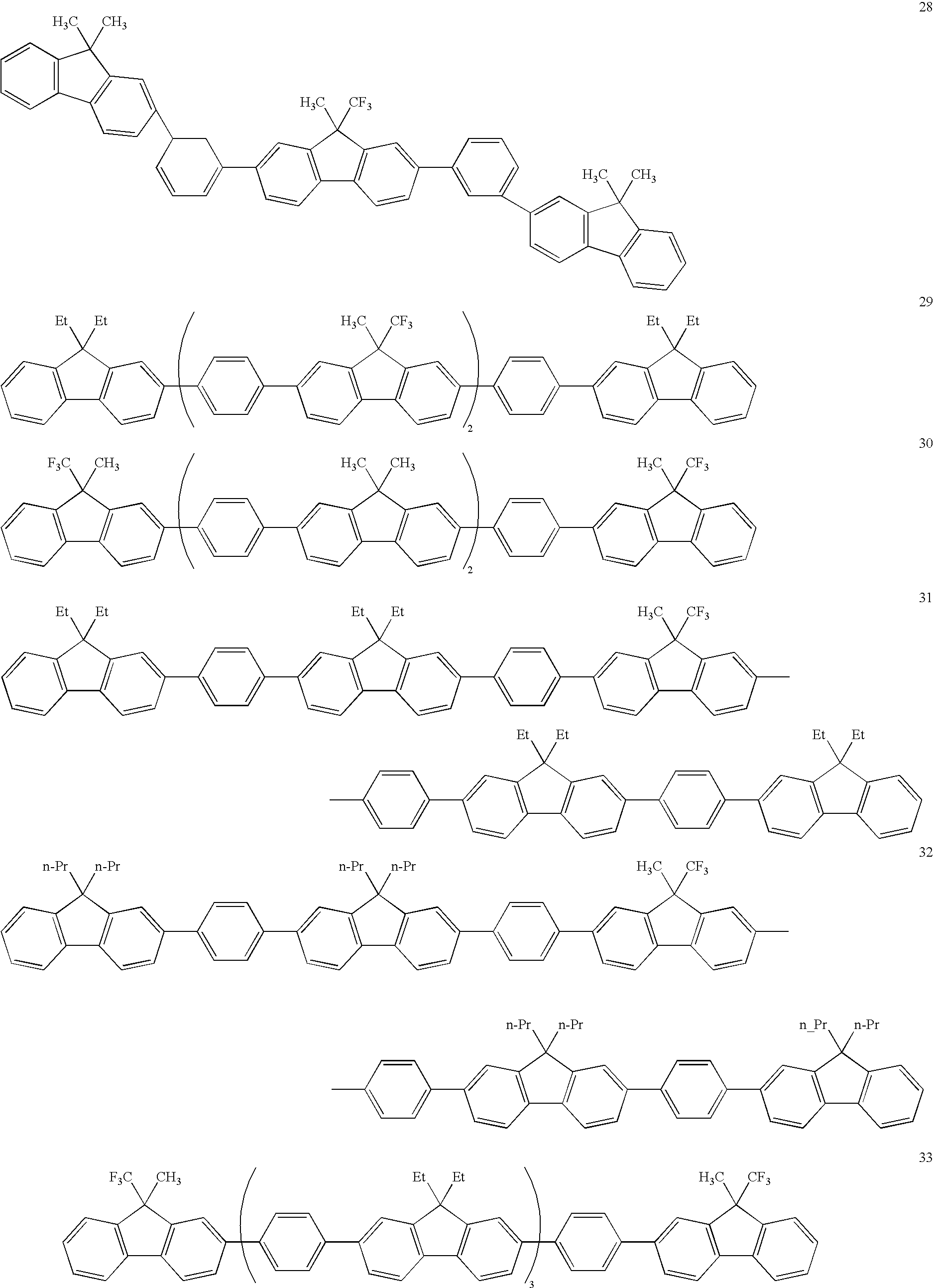

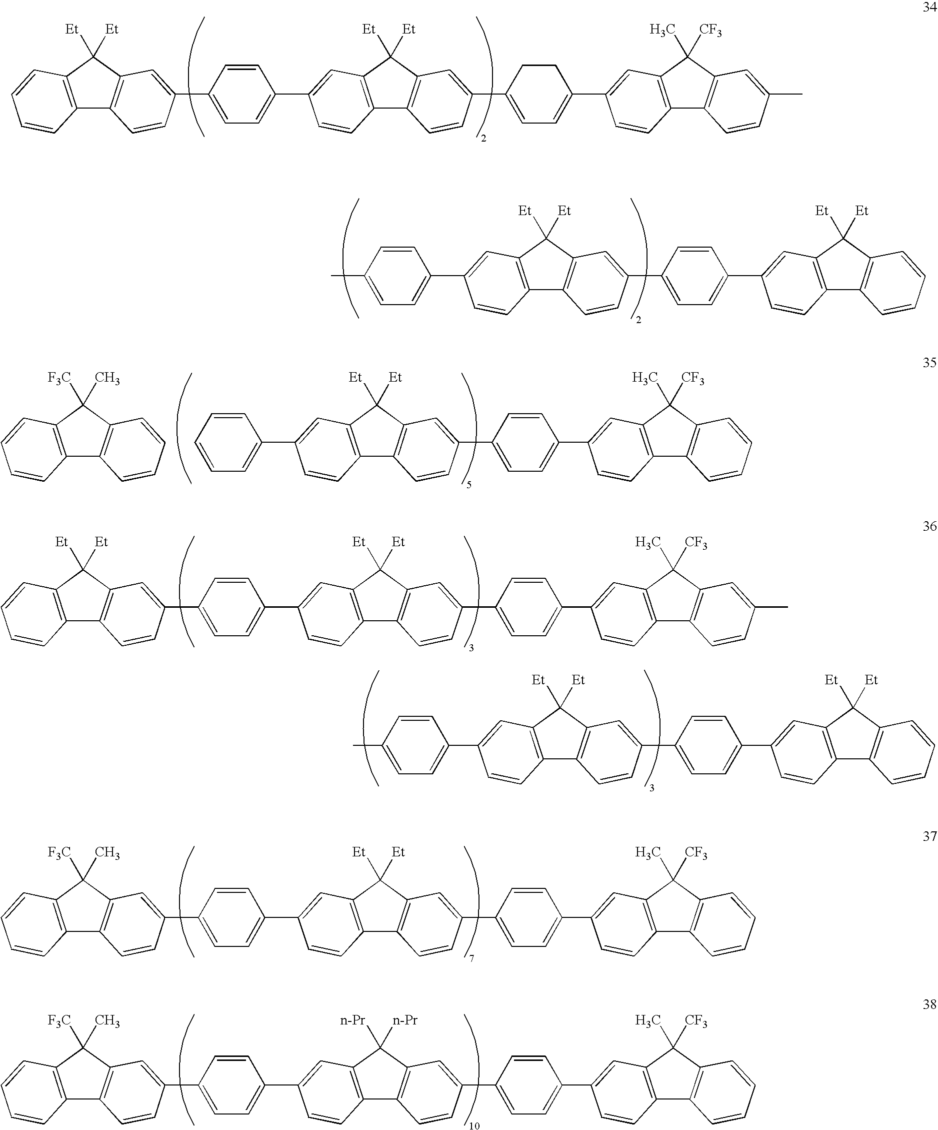

The fluorene compound according to the present invention can be synthesized by generally known methods. For example, the compound can be synthesized by the Suzuki Coupling method using a Palladium catalyst (e.g., Chem. Rev., 95, 2457, 1995), the Yamamoto method using a nickel catalyst (e.g., Bull. Chem. Soc. Jpn. 51, 2091, 1978), and the like.

The fluorene compound according to the present invention is superior in light-emitting property and durability to the conventional compounds, and is useful as an organic-compound-containing layer of an organic light-emitting device, especially as a light-emitting layer. Further, a layer formed by vacuum deposition or solution coating using the fluorene compound according to the present invention is less susceptible to crystallization and is excellent in durability over time.

Next, the organic light-emitting device of the present invention will be described in detail below.

The organic light-emitting device according to the present invention comprises a pair of electrodes including an anode and a cathode, and an organic compound layer provided between the pair of electrodes, wherein the organic compound layer comprises at least one of the compounds according to the present invention.

In the organic light-emitting device of the present invention, it is preferred that at least a light-emitting layer of layer(s) containing organic compound(s) contains at least one of the above-mentioned fluorene compounds.

The layer containing the fluorene compound according to the present invention is formed by a method such as vacuum deposition or solution coating between an anode and a cathode. A fluorene compound having 5 or more fluorene rings in a molecule according to the present invention tends to have a higher sublimation temperature, so that the solution coating is preferably used.

It is preferred that the layer containing the fluorene compound according to the present invention is formed in a thin film with a thickness of generally 10 μm or less, preferably 0.5 μm or less, and more preferably 0.01 μm or more and 0.5 μm or less.

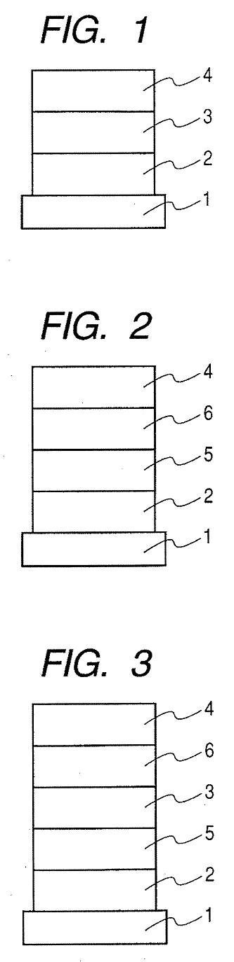

FIGS. 1, 2, 3, 4, 5, 6 and 7 illustrate preferable examples of the organic light-emitting device according to the present invention.

Here, the reference numerals in the figures will be explained.

Reference numeral 1 denotes a substrate, reference numeral 2 denotes an anode, reference numeral 3 denotes a light-emitting layer, reference numeral 4 denotes a cathode, reference numeral 5 denotes a hole-transporting layer, reference numeral 6 denotes an electron-transporting layer, reference numeral 7 denotes a hole injection layer, reference numeral 8 denotes an electron injection layer, and reference numeral 9 denotes a hole/exciton blocking layer.

FIG. 1 is a cross-sectional view showing an example of the organic light-emitting device according to the present invention. In FIG. 1, the device has a configuration in which an anode 2, a light-emitting layer 3, and a cathode 4 are provided sequentially on a substrate 1. A light-emitting device with this configuration is advantageous when the light-emitting material itself has all of hole transportability, electron transportability, and light-emitting property, or when compounds, respectively, having these characteristics are used in combination.

FIG. 2 is a cross-sectional view showing another example of the organic light-emitting device according to the present invention. In FIG. 2, the device has a configuration such that an anode 2, a hole-transporting layer 5, an electron-transporting layer 6, and a cathode 4 are formed sequentially on a substrate 1. A light-emitting device with this configuration is advantageous when a light-emitting material having either or both of hole transportability and electron transportability is used for the respective layers, in combination with a hole-transporting material having no light-emitting property or an electron-transporting material having no light-emitting property. In addition, in this case, either one of the hole-transporting layer 5 and the electron-transporting layer 6 also serves as the light-emitting layer.

FIG. 3 is a cross-sectional view showing still another example of the organic light-emitting device according to the present invention. In FIG. 3, the device has a configuration in which an anode 2, a hole-transporting layer 5, a light-emitting layer 3, an electron-transporting layer 6, and a cathode 4 are formed sequentially on a substrate 1. With this configuration, the carrier-transporting function and the light-emitting function are separated from each other, and compounds, respectively, having hole-transporting property, electron-transporting property, and light-emitting property can be used appropriately in combination, so that the degree of freedom in selecting materials greatly increases. Further, since various kinds of compounds having different emission wavelengths can be used, a variety of emission wavelengths can be achieved. Moreover, carriers or excitons can be effectively confined in the light-emitting layer at the middle portion, to thereby increase the emission efficiency.

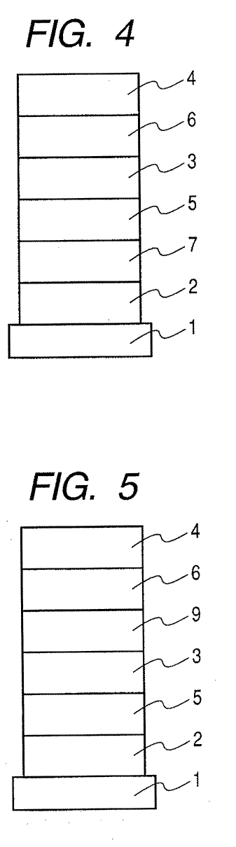

FIG. 4 is a cross-sectional view showing yet another example of the organic light-emitting device according to the present invention. In FIG. 4, as compared with FIG. 3, the device is constructed such that a hole injection layer 7 is provided on the anode side, which is effective for improving adhesion between the anode 2 and the hole-transporting layer 5 or improving the hole injection property, thus being effective for reducing the driving voltage.

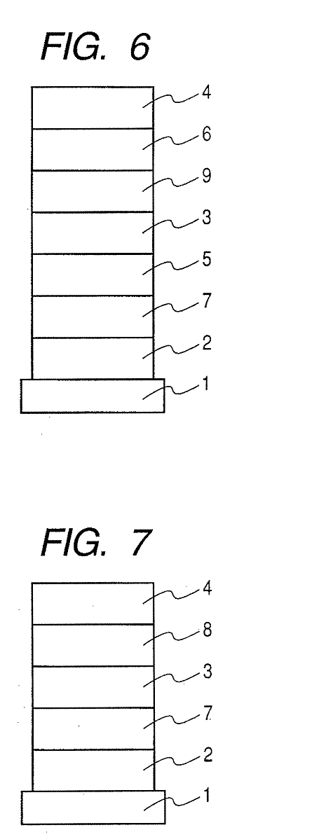

FIGS. 5 and 6 are cross-sectional views showing yet still other examples of the organic light-emitting device according to the present invention. In each of FIGS. 5 and 6, as compared with FIGS. 3 and 4, the device is constructed such that a layer (a hole/exciton blocking layer 9) serving to prevent holes or excitons from passing through toward the cathode 4 is provided between the light-emitting layer 3 and the electron-transporting layer 6. Using a compound having an extremely high ionization potential for the hole/exciton blocking layer 8 is effective for improving the emission efficiency.

FIG. 7 is a cross-sectional view showing yet again another example of the organic light-emitting device according to the present invention. In FIG. 7, the device has a configuration in which an anode 2, a hole injection layer 7, a light-emitting layer 3, an electron injection layer 8, and a cathode 4 are formed sequentially on a substrate 1.

It is to be noted that FIGS. 1, 2, 3, 4, 5, 6 and 7 merely show very basic device configurations, and that the structure of the organic light-emitting device according to the present invention is not limited thereto. For example, it is possible to adopt various layer configurations, such as one in which an insulating layer is provided at an interface between an electrode and an organic layer, one in which an adhesive layer or an interference layer is provided, or one in which a hole-transporting layer is composed of two layers with different ionization potentials.

The fluorene compound according to the present invention is superior in light-emitting property and durability to the conventional compounds and can be used in any one of the configurations illustrated in FIGS. 1, 2, 3, 4, 5, 6 and 7.

In particular, an organic layer using the fluorene compound according to the present invention is useful as a light-emitting layer, and a layer formed by vacuum deposition or solution coating using the fluorene compound according to the present invention is less susceptible to crystallization and is excellent in durability over time.

The fluorene compound according to the present invention can also be used in combination with a hitherto known hole-transporting compound, light-emitting compound, electron-transporting compound, or the like. In this case, the content of the fluorene compound according to the present invention is preferably 1 wt. % or more and 50 wt. % or less, and more preferably 1 wt. % or more and 30 wt. % or less.

Examples of such compounds will be shown below.

<Hole-Transporting Compound>

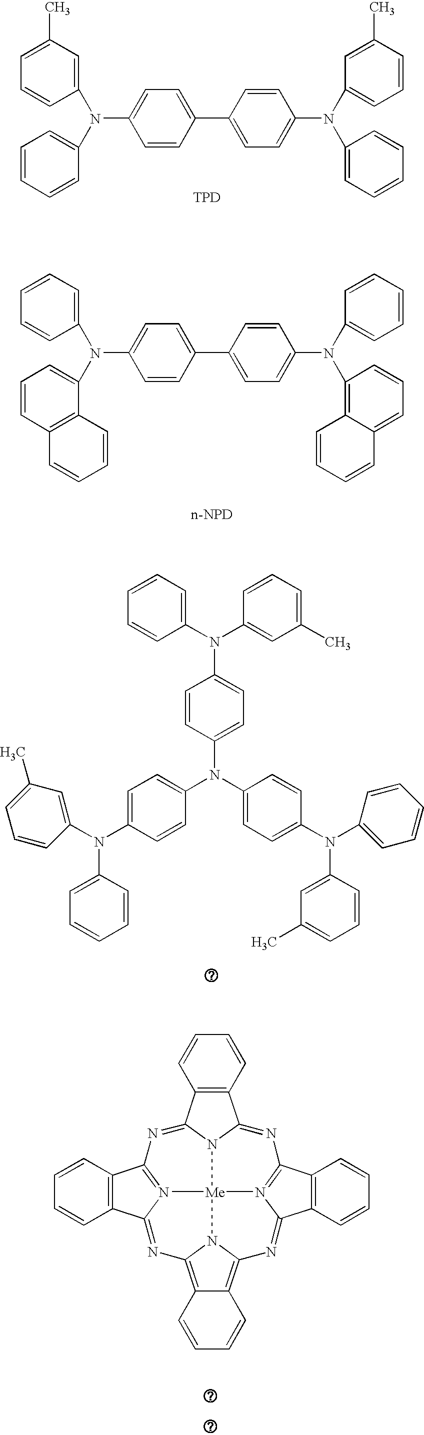

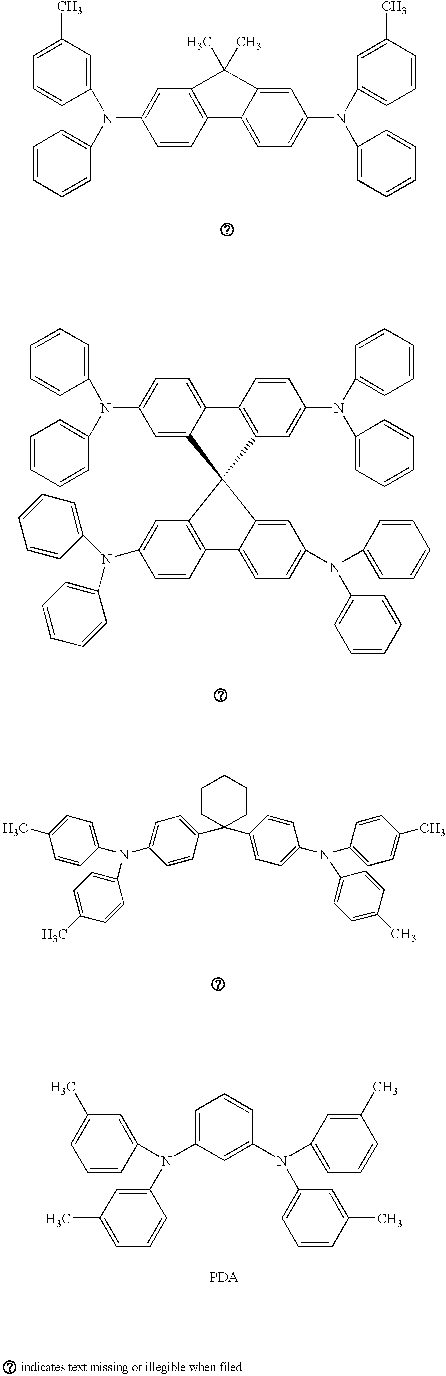

<Electron-Transporting, Light-Emitting Compound>

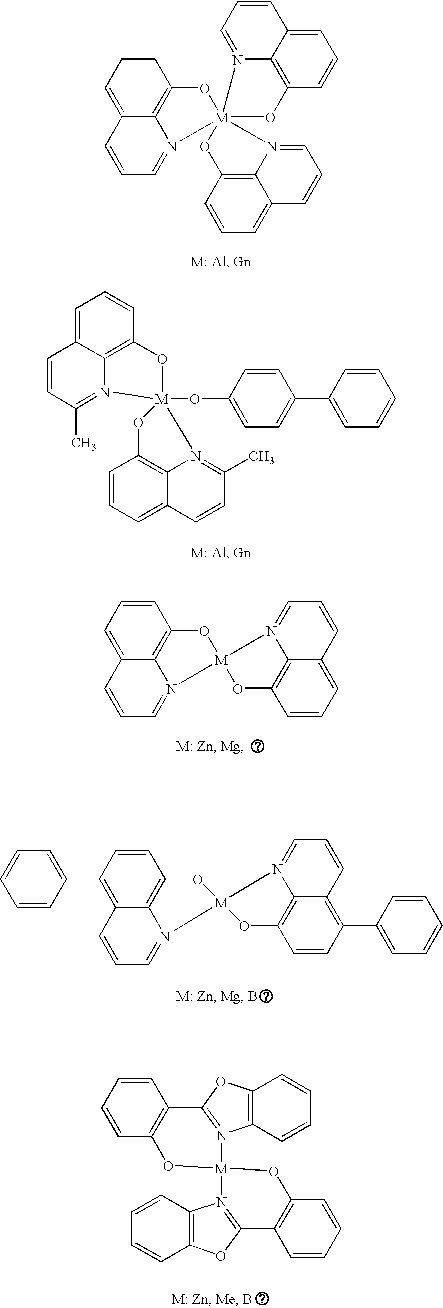

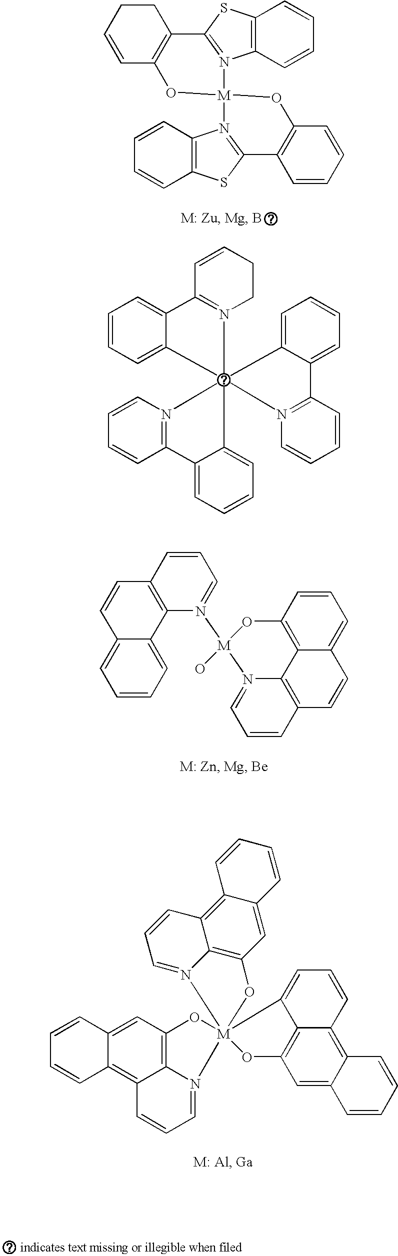

<Light-Emitting Compound>

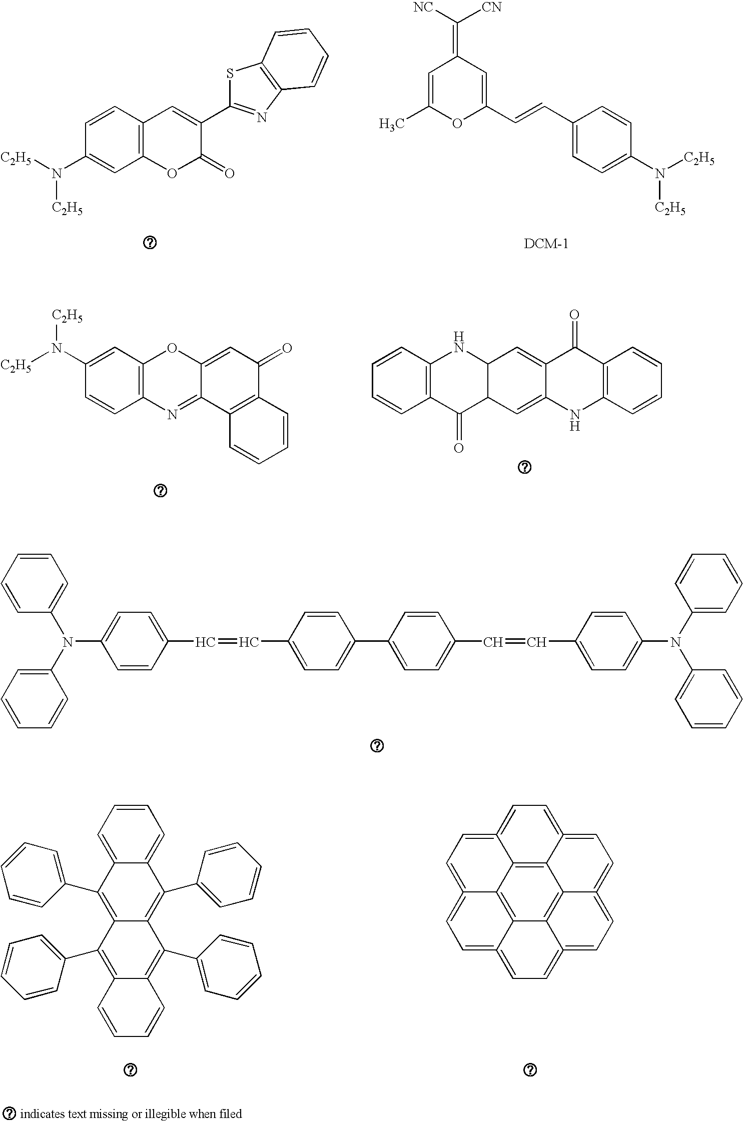

<Light-Emitting Layer Matrix Compound and Electron-Transporting Compound>

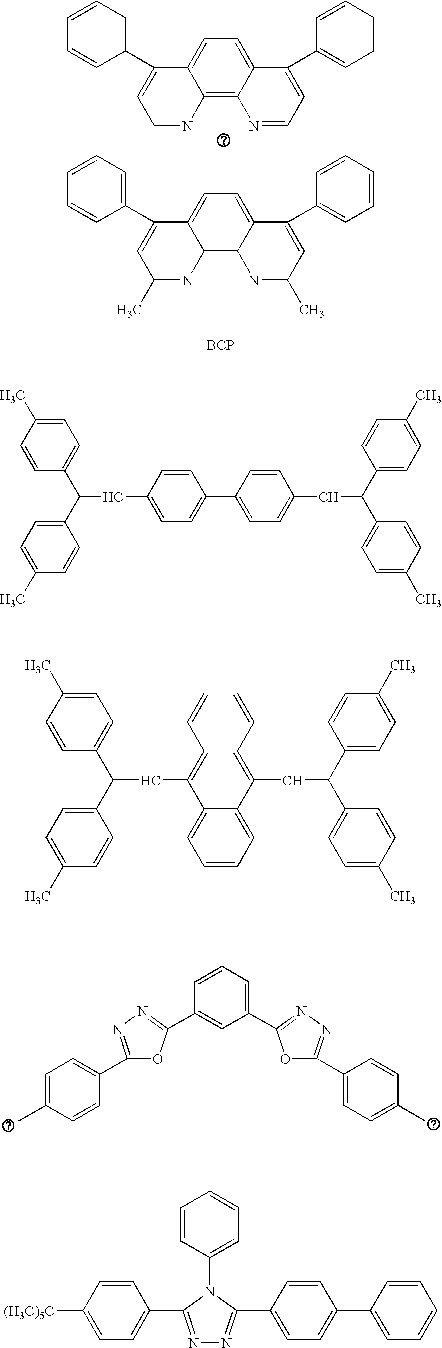

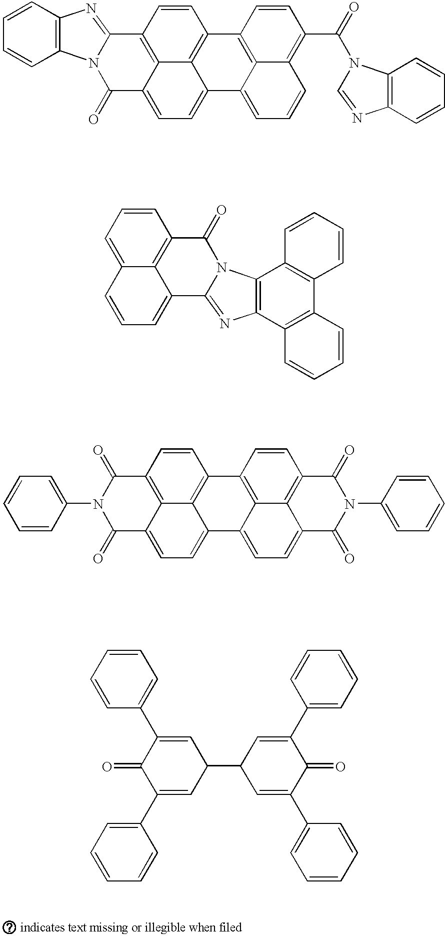

<Polymeric Hole-Transporting Compound>

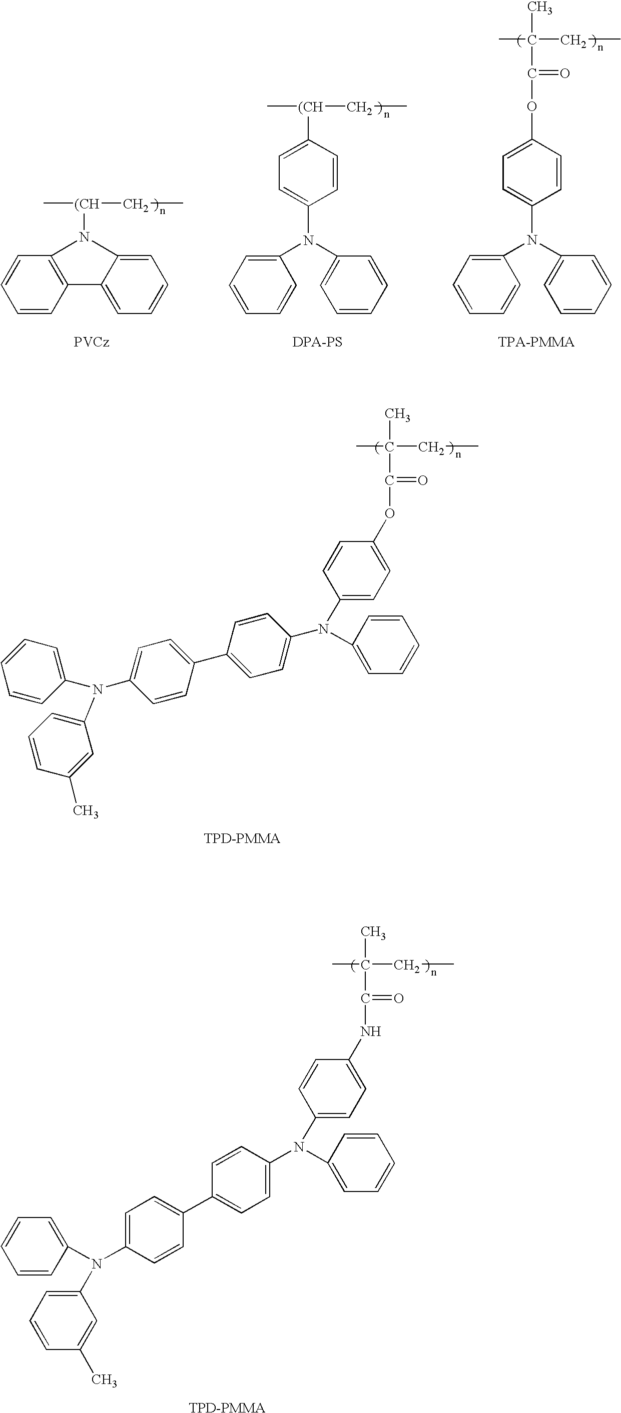

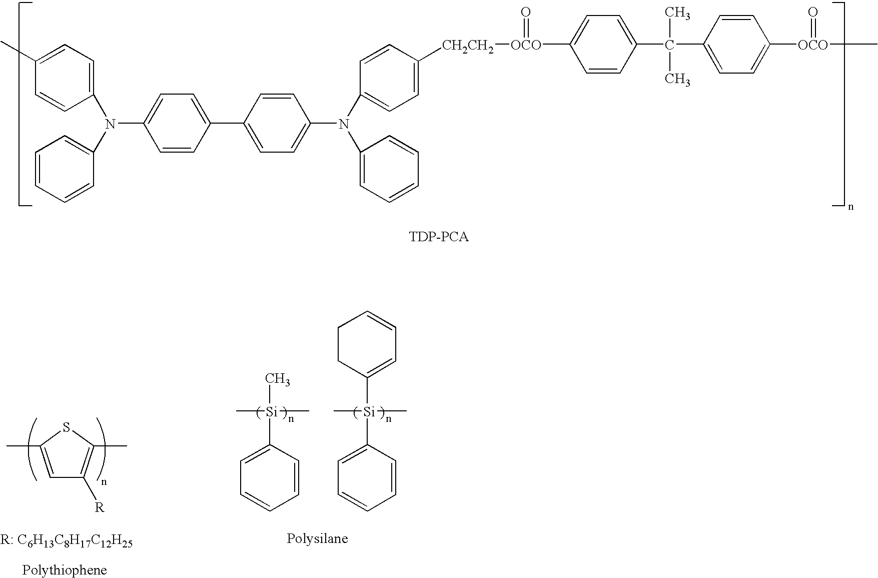

<Polymeric Light-Emitting Compound and Charge-Transporting Compound>

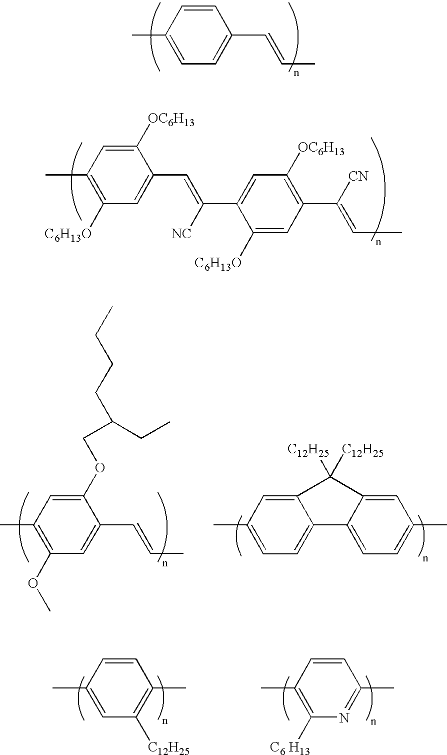

In the organic light-emitting device of the present invention, the layer containing the fluorene compound of the present invention and the other layers containing an organic compound are formed as a thin film generally by using a vacuum deposition method or a coating method of applying such organic compound dissolved in a suitable solvent. As the coating method, there are included a spin coating method, a slit coating method, a printing method, an ink jet method, and a spraying method.

Particularly, when the film is formed by the coating method, the film can be formed by additionally using a suitable binder resin.

The above-mentioned binder resin can be selected from a wide range of binding resins, and includes, for instance, polyvinylcarbazole resin, polycarbonate resin, polyester resin, polyarylate resin, polystyrene resin, acrylic resin, methacrylic resin, butyral resin, polyvinylacetal resin, diallylphthalate resin, phenolic resin, epoxy resin, silicone resin, polysulfonic resin and urea resin, but is not limited thereto. In addition, the binder resin may be singly used, or be used in combination as a copolymer.

An anode material used preferably has as large a work function as possible, and includes, for instance, an elemental metal such as gold, silver, platinum, nickel, palladium, cobalt, selenium, and vanadium, an alloy thereof, and a metal oxide such as stannic oxide, zinc oxide, indium tin oxide (ITO) and indium zinc oxide. Further, a conductive polymer such as polyaniline, polypyrrole, polythiophene, and polyphenylene sulfide can be employed. These electrode materials can be used singly or in combination.

On the other hand, a cathode material used preferably has a low work function, and includes, for instance an elemental metal such as lithium, sodium, potassium, cesium, calcium, magnesium, aluminum, indium, silver, lead, tin, and chromium; an alloy made of a plurality of the above metals; or a salt thereof. A metal oxide such as indium tin oxide (ITO) can be also used. In addition, the cathode may be either of a single layer configuration or of a multilayer configuration.

A substrate used in the present invention is not particularly limited, but an opaque substrate such as a metal substrate and a ceramic substrate or a transparent substrate such as glass, quartz, and a plastic sheet is used. Further, it is also possible to employ, for a substrate, a color filter film, a fluorescent color conversion filter film and a dielectric reflective film to thereby control the emission color.

Incidentally, after a device has been produced, a protective layer or an encapsulation layer may further be provided, for the purpose of preventing contact with oxygen or moisture. Examples of such a protective layer include a diamond thin film; a film of an inorganic material such as a metal oxide and a metal nitride; a film of a polymer such as a fluororesin, poly-p-xylene, polyethylene, silicone resin, and polystyrene resin; and further a film of a photocurable resin. Further, the produced device may also be covered with glass, a gas-impermeable film and a metal, or be packaged with a suitable encapsulation resin.

Next, application of the compound of the present invention to an ink composition will be described below.

The ink composition contains at least one of the fluorene compounds of the present invention. By using the ink composition, an organic compound layer of an organic light-emitting device, especially a light-emitting layer can be formed by the linear drawing method and a device with a large area which is relatively less expensive can easily be produced. In particular, since a fluorene compound having 5 or more fluorene rings in a molecule according to the present invention tends to have a higher sublimation temperature, it is appropriate to dissolve the compound in a solvent and use the compound in the form of an ink composition.

As the solvent, there are included toluene, xylene, mesitylene, dioxane, tetralin, methylnaphthalene, tetrahydrofuran, and bis(2-methoxyethyl)ether (Diglyme).

In addition to the fluorene compound according to the present invention, the composition may also contain, for example, the above-mentioned hitherto known hole-transporting compound, light-emitting compound, and electron-transporting compound.

The content of the fluorene compound according to the present invention in the ink composition is preferably 0.1 wt. % or more and 10 wt. % or less, and more preferably 0.1 wt. % or more and 3 wt. % or less.

### EXAMPLES

The present invention will be now described more in detail below with reference to examples, but the present invention is not limited to the examples.

<Synthesis of Intermediates>

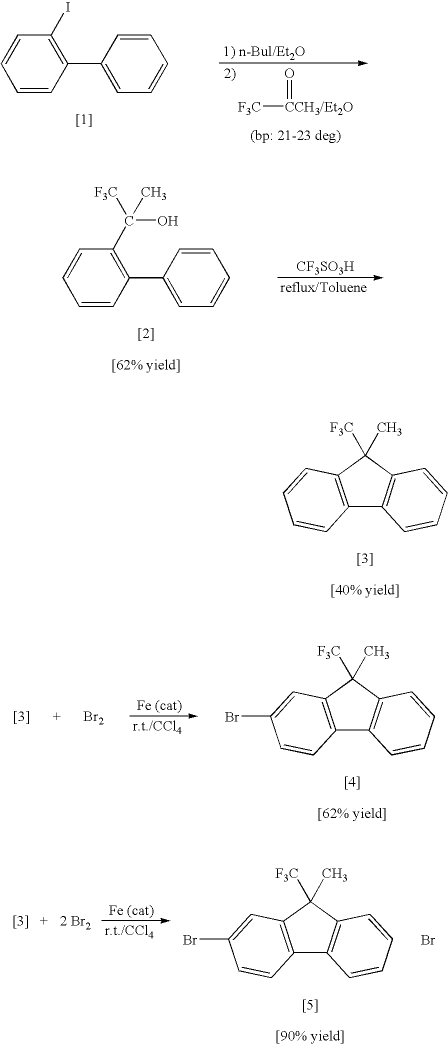

20 g (71.4 mmole) of 2-iodobiphenyl [1] and 200 ml of diethyl ether were charged into a 500-ml three neck flask, and 51 ml (78.5 mmole) of 15% n-butyllithium/hexane solution was added dropwise thereto at −78° C. under nitrogen atmosphere. After the whole was stirred at −78° C. for 1 hour, 10.1 ml (107 mmole) of 1,1,1-trifluoroacetone was added dropwise thereto, and the whole was gradually warmed to room temperature and then stirred for 8 hours. After the completion of the reaction, the organic layer was extracted with ethyl acetate, dried with anhydrous sodium sulfate, and then purified by silica gel column chromatography (hexane+toluene mixed developing solvent) to give 11.8 g of adduct [2] (transparent liquid) (yield: 62%).

10 g (37.6 mmole) of the adduct [2] and 150 ml of toluene were charged into a 300-ml three neck flask, and 1.9 g (12.5 mmole) of trifluoromethanesulfonate was added dropwise thereto at room temperature, followed by reflux for 8 hours. After the completion of the reaction, water was added thereto, and the organic layer was extracted with ethyl acetate, dried with anhydrous sodium sulfate, and then purified by silica gel column chromatography (hexane+toluene mixed developing solvent) to give 3.7 g of fluorene intermediate [3] (transparent liquid) (yield: 40%).

1.9 g (7.7 mmole) of the fluorene intermediate [3] and 40 ml of carbon tetrachloride were charged into a 100-ml three neck flask, a catalytic amount of iron powder was added thereto at room temperature, and then a solution of 1.4 g (8.5 mmole) of bromine and 5 ml of carbon tetrachloride was added dropwise thereto. After stirring for 5 hours at room temperature, water was added thereto, and the organic layer was extracted with chloroform, dried with anhydrous sodium sulfate, and then purified by silica gel column chromatography (hexane developing solvent) to give 2.1 g of monobromofluorene intermediate [4] (transparent liquid) (yield: 82%).

1.9 g (7.7 mmole) of the fluorene intermediate [3] and 40 ml of carbon tetrachloride were charged into a 100-ml three neck flask, a catalytic amount of iron powder was added thereto at room temperature, and then a solution of 2.6 g (16.2 mmole) of bromine and 5 ml of carbon tetrachloride was added dropwise thereto. After stirring for 8 hours at room temperature, water was added thereto, and the organic layer was extracted with chloroform, dried with anhydrous sodium sulfate, and then purified by silica gel column chromatography (hexane developing solvent) to give 2.8 g of dibromofluorene intermediate [5] (white crystal) (yield: 90%).

<Synthesis Example 1> Synthesis of Exemplary Compound No. 11

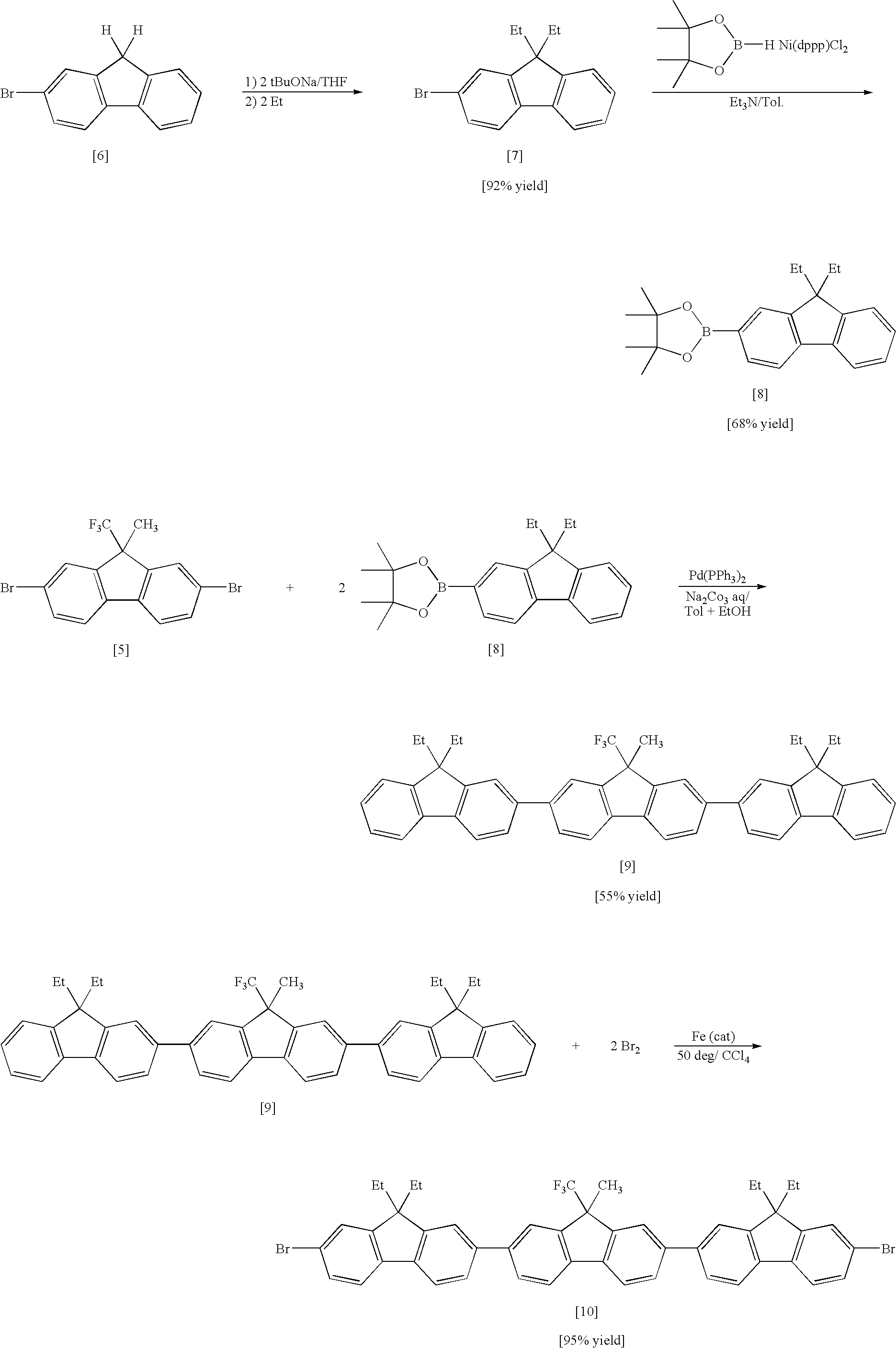

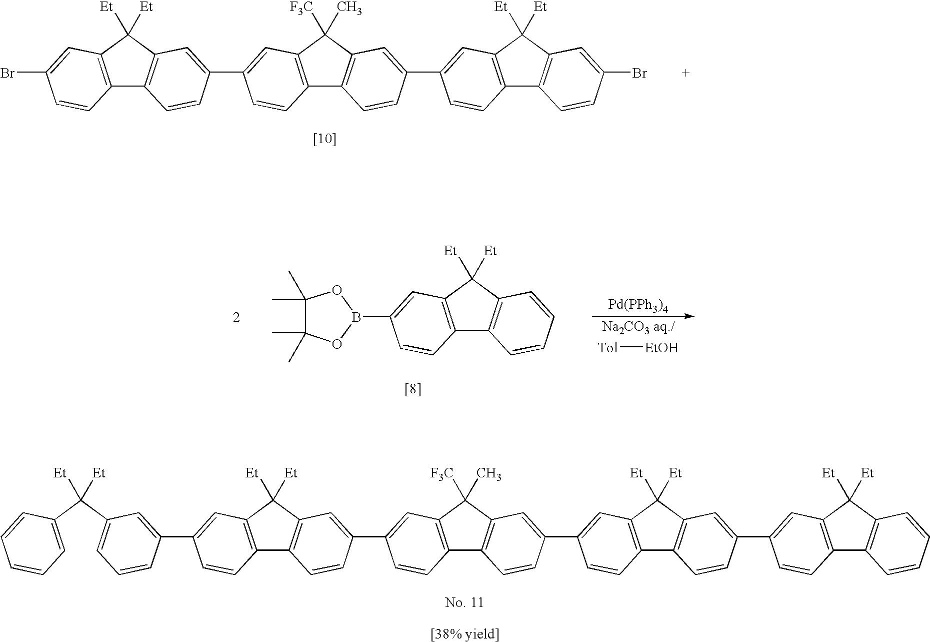

(1) Synthesis of Pinacolborane Intermediate

25 g (102 mmole) of 2-bromofluorene [6] and 250 ml of THF were charged into a 500-ml three neck flask, and 22.5 g (235 mmole) of tert-butoxysodium was added dropwise thereto at 0° C. under nitrogen atmosphere. After the whole was stirred at that temperature for 30 minutes, 40 g (255 mmole) of iodoethane was added dropwise thereto, and the whole was gradually warmed to room temperature and then stirred for 8 hours. After the completion of the reaction, water was poured thereinto, and the organic layer was extracted with chloroform, dried with anhydrous sodium sulfate, and then purified by silica gel column chromatography (hexane developing solvent) to give 28.2 g of monobromo intermediate [7] (transparent liquid) (yield: 92%).

The following components were charged into a 500-ml three neck flask and refluxed for 24 hours.

Monobromo intermediate [7] 23 g (76.3 mmole)

Pinacolborane 22 g (172 mmole)

(Diphenylphosphinopropane)dichloronickel 4.1 g (7.6 mmole)

Triethylamine 50 ml

Toluene 200 ml

After the completion of the reaction, water was poured thereinto, and the organic layer was extracted with ethyl acetate, dried with anhydrous sodium sulfate, and then purified by silica gel column chromatography (hexane+toluene mixed developing solvent) to give 18.0 g of pinacolborane intermediate [8] (transparent liquid) (yield: 68%).

(2) Synthesis of Dibromotrisfluorene Intermediate

1.5 g (3.69 mmole) of the dibromofluorene intermediate [5], 2.8 g (8.13 mmole) of the pinacolborane intermediate [8], 10 ml of toluene and 50 ml of ethanol were charged into a 300-ml three neck flask. An aqueous solution of 14 g of sodium carbonate and 70 ml of water was added dropwise thereto with stirring at room temperature under nitrogen atmosphere, and then 0.43 g (0.37 mmole) of tetraxis(triphenylphosphine)palladium(0) was added. The whole was stirred at room temperature for 30 minutes, then warmed to 77° C. and stirred for 3 hours. After the completion of the reaction, the organic layer was extracted with chloroform, dried with anhydrous sodium sulfate, and then purified by silica gel column chromatography (hexane+toluene mixed developing solvent) to give 1.4 g of trisfluorene intermediate [9] (white crystal) (yield: 55%).

1.0 g (1.5 mmole) of the trisfluorene intermediate [9] and 20 ml of carbon tetrachloride were charged into a 50-ml three neck flask, a catalytic amount of iron powder was added thereto at room temperature, and then a solution of 0.5 g (3.1 mmole) of bromine and 5 ml of carbon tetrachloride was added dropwise thereto. After stirring for 8 hours at room temperature, water was added thereto, and the organic layer was extracted with chloroform, dried with anhydrous sodium sulfate, and then purified by silica gel column chromatography (hexane+toluene mixed developing solvent) to give 1.2 g of dibromotrisfluorene intermediate [10] (white crystal) (yield: 95%).

(3) Synthesis of Exemplary Compound No. 11

0.5 g (0.59 mmole) of the dibromotrisfluorene intermediate [10], 0.5 g (2.4 mmole) of the pinacolborane intermediate [8], 60 ml of toluene and 30 ml of ethanol were charged into a 200-ml three neck flask. An aqueous solution of 2.4 g of sodium carbonate and 12 ml of water was added dropwise thereto with stirring at room temperature under nitrogen atmosphere, and then 0.07 g (0.06 mmole) of tetraxis(triphenylphosphine)palladium(0) was added. The whole was stirred at room temperature for 30 minutes, then warmed to 77° C. and stirred for 5 hours. After the completion of the reaction, the organic layer was extracted with chloroform, dried with anhydrous sodium sulfate, and then purified by silica gel column chromatography (hexane+toluene mixed developing solvent) to give 0.25 g of Exemplary Compound No. 11 (white amorphous substance) (yield: 38%).

<Synthesis Example 2> Synthesis of Exemplary Compound No. 13

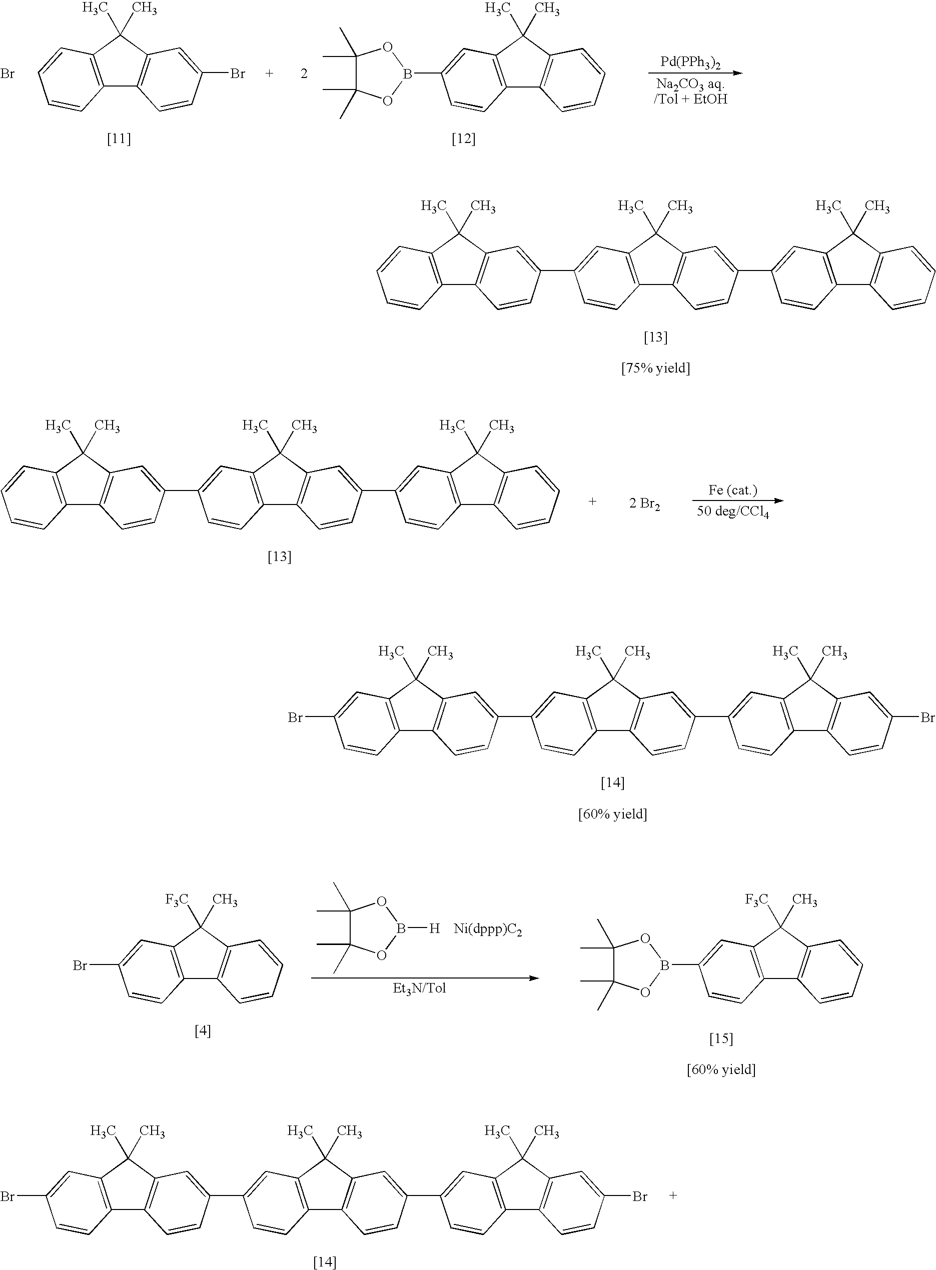

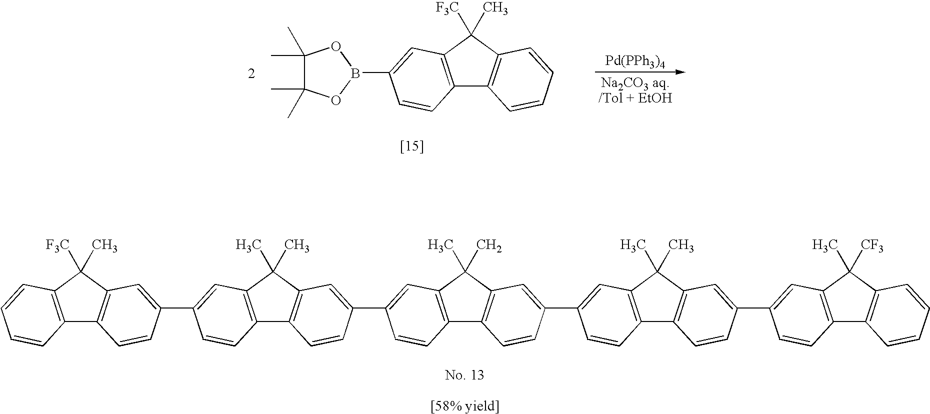

(1) Synthesis of Dibromotrisfluorene Intermediate and Pinacolborane Intermediate

Dibromotrisfluorene intermediate 2 [14] and pinacolborane intermediate 2 [15] were obtained through a synthesis route similar to that of Synthesis Example 1.

Synthesis of Exemplary Compound No. 13

0.5 g (0.68 mmole) of the dibromotrisfluorene intermediate 2 [14], 0.64 g (1.7 mmole) of the pinacolborane intermediate 2 [15], 60 ml of toluene and 30 ml of ethanol were charged into a 200-ml three neck flask. An aqueous solution of 2.4 g of sodium carbonate and 12 ml of water was added dropwise thereto with stirring at room temperature under nitrogen atmosphere, and then 0.07 g (0.06 mmole) of tetraxis(triphenylphosphine)palladium(0) was added. The whole was stirred at room temperature for 30 minutes, then warmed to 77° C. and stirred for 5 hours. After the completion of the reaction, the organic layer was extracted with chloroform, dried with anhydrous sodium sulfate, and then purified by silica gel column chromatography (hexane+toluene mixed developing solvent) to give 0.43 g of Exemplary Compound No. 13 (white crystal) (yield: 58%).

### Example 1

A device with the structure shown in FIG. 7 was produced.

A transparent conductive support substrate was prepared which had a film of indium tin oxide (ITO) with a thickness of 120 nm as the anode 2 formed on a glass plate as the substrate 1 by a sputtering method. The transparent conductive support substrate was ultrasonically cleaned sequentially with acetone and isopropyl alcohol (IPA), subsequently washed with boiled IPA, was then dried, was further cleaned with UV/ozone, and was used.

On the transparent conductive support substrate, a film of poly(3,4-ethylenedioxythiophene) (Baytron P AI-4083 (trade name); manufactured by Bayer) was made in a thickness of 50 nm by spin coating to form the hole injection layer 7.

Further, a film was made in a thickness of 60 nm by spin coating of a 2 wt. % toluene solution of Exemplary Compound No. 11 to form the light-emitting layer.

Next, as the electron injection layer 8, calcium was used to form a metal layer film in a thickness of 1 nm by vacuum deposition on the organic layers. The vacuum degree during the deposition was 1.0×10⁻⁴ Pa and the film forming rate was 0.1 nm/sec.

Further, as the cathode 4, an aluminum layer with a thickness of 150 nm was formed by vacuum deposition. The vacuum degree during the deposition was 1.0×10⁻⁴ Pa and the film forming rate was 0.1 to 1.2 nm/sec.

Moreover, covering with a protective glass plate was performed in nitrogen atmosphere, followed by encapsulation with an acrylic resin adhesive.

When a DC voltage of 5 V was applied to the thus obtained device with the ITO electrode (anode 2) being connected to a positive electrode and the Al electrode (cathode 4) being connected to a negative electrode, a current flowed through the device at a current density of 35 mA/cm² and emission of a blue light was observed with a luminance of 2,100 cd/m².

Furthermore, when a voltage was applied to the device for 50 hours so that the current density was kept at 5.0 mA/cm², the device emitted light at a luminance of 550 cd/m² in an early stage and at 450 cd/m² after the elapse of the 50 hours, which meant that the luminance degradation was small.

### Examples 2-3 and 5-20

Devices were produced by following the same procedure as in Example 1 with the exception that the exemplified compounds shown in Table 1 were used in place of Exemplified Compound No. 11 used in Example 1, and the same evaluation as in Example 1 was performed thereto. The results are shown in Table 1.

### Comparative Examples 1-3

Devices were produced by following the same procedure as in Example 1 with the exception that the compounds shown in Table 1 were used in place of Exemplified Compound No. 11 used in Example 1, and the same evaluation as in Example 1 was performed thereto. The results are shown in Table 1.

TABLE 1

| | | Initial | | Durability | | |
|---|---|---|---|---|---|---|
| | | Applied | | Current | Initial | Luminance |
| | Exemplary | Voltage | Luminance | Density | Luminance | after 50 |
| | Compound No. | (V) | (cd/m²) | (mA/cm²) | (cd/m²) | hours (cd/m²) |
| Ex. 1 | 11 | 5 | 2100 | 5.0 | 550 | 450 |
| Ex. 2 | 3 | 5 | 1900 | 5.0 | 560 | 420 |
| Ex. 3 | 6 | 5 | 1700 | 5.0 | 520 | 400 |
| Ex. 5 | 13 | 5 | 2400 | 5.0 | 580 | 510 |
| Ex. 6 | 15 | 5 | 1800 | 5.0 | 520 | 400 |
| Ex. 7 | 16 | 5 | 2200 | 5.0 | 500 | 420 |
| Ex. 8 | 18 | 5 | 2400 | 5.0 | 540 | 440 |
| Ex. 9 | 21 | 5 | 2300 | 5.0 | 480 | 450 |
| Ex. 10 | 22 | 5 | 2000 | 5.0 | 480 | 440 |
| Ex. 11 | 25 | 5 | 2200 | 5.0 | 550 | 440 |
| Ex. 12 | 27 | 5 | 2000 | 5.0 | 470 | 400 |
| Ex. 13 | 29 | 5 | 1700 | 5.0 | 490 | 410 |
| Ex. 14 | 32 | 5 | 2300 | 5.0 | 530 | 430 |
| Ex. 15 | 33 | 5 | 2200 | 5.0 | 500 | 420 |
| Ex. 16 | 34 | 5 | 1600 | 5.0 | 510 | 420 |
| Ex. 17 | 35 | 5 | 1900 | 5.0 | 500 | 400 |
| Ex. 18 | 36 | 5 | 1700 | 5.0 | 530 | 420 |
| Ex. 19 | 37 | 5 | 1700 | 5.0 | 540 | 410 |
| Ex. 20 | 38 | 5 | 1800 | 5.0 | 500 | 430 |
| Comp. | Comp. Compd. | 5 | 1300 | 5.0 | 420 | 250 |
| Ex. 1 | 1 | | | | | |
| Comp. | Comp. Compd. | 5 | 800 | 5.0 | 350 | 150 |
| Ex. 2 | 2 | | | | | |
| Comp. | Comp. Compd. | 5 | 1200 | 5.0 | 430 | 120 |
| Ex. 3 | 3 | | | | | |

(Comparative Compound No. 1)
Poly(9,9-dioctyl)fluorene (manufactured by American
Dye Source Inc.; Mw = 80,000)

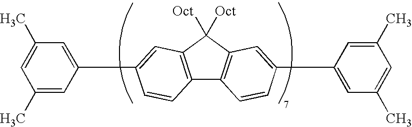

(Comparative Compound No. 2)

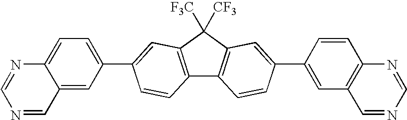

(Comparative Compound No. 3)

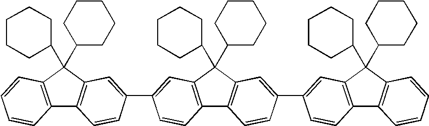

### Example 21

A device was produced by following the same procedure as in Example 1 with the exception that a film with a thickness of 60 nm was made by spin coating of a toluene solution which contained 0.2 wt. % of the Ir complex represented by the following structural formula and 2 wt. % of Exemplary Compound No. 4 to thereby form the light-emitting layer 3.

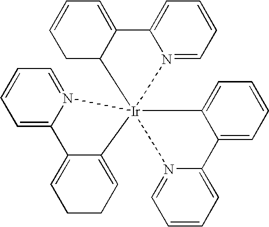

When a DC voltage of 5 V was applied to the thus obtained device with the ITO electrode (anode 2) being connected to a positive electrode and the Al electrode (cathode 4) being connected to a negative electrode, a current flowed through the device at a current density of 20 mA/cm² and emission of a green light was observed with a luminance of 1,600 cd/m².

Furthermore, when a voltage was applied to the device for 50 hours so that the current density was kept at 5.0 mA/cm², the device emitted light at a luminance of 600 cd/m² in an early stage and at 520 cd/m² after the elapse of the 50 hours, which meant that the luminance degradation was small.

### Examples 22-31

Devices were produced by following the same procedure as in Example 21 with the exception that the compounds shown in Table 2 were used in place of Exemplified Compound No. 4 used in Example 21, and the same evaluation as in Example 21 was performed thereto. The results are shown in Table 2.

### Comparative Examples 4-6

Devices were produced by following the same procedure as in Example 21 with the exception that Comparative Compound 1, 2, and 3 were used in place of Exemplified Compound No. 4 used in Example 21, and the same evaluation as in Example 21 was performed thereto. The results are shown in Table 2.

TABLE 2

| | | | | Durability | | |
|---|---|---|---|---|---|---|
| | | Initial | | | | Luminance |
| | | Applied | | Current | Initial | after 50 |
| | Exemplary | Voltage | Luminance | Density | Luminance | hours |
| | Compound No. | (V) | (cd/m²) | (mA/cm²) | (cd/m²) | (cd/m²) |
| Ex. 21 | 4 | 5 | 1600 | 5.0 | 600 | 520 |
| Ex. 22 | 7 | 5 | 1300 | 5.0 | 560 | 440 |
| Ex. 23 | 12 | 5 | 1800 | 5.0 | 530 | 540 |
| Ex. 24 | 14 | 5 | 1800 | 5.0 | 500 | 450 |
| Ex. 25 | 17 | 5 | 1400 | 5.0 | 480 | 420 |
| Ex. 26 | 19 | 5 | 1600 | 5.0 | 500 | 400 |
| Ex. 27 | 20 | 5 | 1500 | 5.0 | 530 | 420 |
| Ex. 28 | 23 | 5 | 1400 | 5.0 | 510 | 430 |
| Ex. 29 | 24 | 5 | 1500 | 5.0 | 490 | 420 |
| Ex. 30 | 30 | 5 | 1500 | 5.0 | 480 | 410 |
| Ex. 31 | 31 | 5 | 1800 | 5.0 | 590 | 540 |
| Comp. | Comp. Compd. 1 | 5 | 1100 | 5.0 | 400 | 290 |
| Ex. 4 | | | | | | |
| Comp. | Comp. Compd. 2 | 5 | 750 | 5.0 | 320 | 150 |
| Ex. 5 | | | | | | |
| Comp. | Comp. Compd. 3 | 5 | 1000 | 5.0 | 420 | 100 |
| Ex. 6 | | | | | | |

### Example 32

A device with the structure shown in FIG. 2 was produced.

On a transparent conductive support substrate similar to that prepared in Example 1, a film was made in a thickness of 30 nm by spin coating using a 2 wt. % of chloroform solution of a compound represented by the following formula to form the hole transporting layer 5.

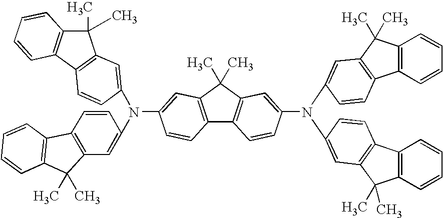

Further, a film was made in a thickness of 50 nm by vacuum deposition of Exemplary Compound No. 1 to form the light-emitting layer 3. The vacuum degree during the deposition was 1.0×10⁻⁴ Pa and the film forming rate was 0.2 to 0.3 nm/sec.

Further, as the cathode 4, a deposition material made of aluminum and lithium (lithium concentration: 1 atomic %) was used to form a metal layer film in a thickness of 50 nm by vacuum deposition on the organic layers, and an aluminum layer with a thickness of 150 nm was formed thereon by vacuum deposition. The vacuum degree during the deposition was 1.0×10⁻⁴ Pa and the film forming rate was 1.0 to 1.2 nm/sec.

Moreover, covering and encapsulation were performed in the same manner as in Example 1.

When a DC voltage of 5 V was applied to the thus obtained device with the ITO electrode (anode 2) being connected to a positive electrode and the Al electrode (cathode 4) being connected to a negative electrode, a current flowed through the device at a current density of 60 mA/cm² and emission of a blue light was observed with a luminance of 1,500 cd/m².

Furthermore, when a voltage was applied to the device for 100 hours so that the current density was kept at 20.0 mA/cm², the device emitted light at a luminance of 580 cd/m² in an early stage and at 500 cd/m² after the elapse of the 100 hours, which meant that the luminance degradation was small.

### Examples 33-38

Devices were produced by following the same procedure as in Example 32 with the exception that the compounds shown in Table 3 were used in place of Exemplified Compound No. 1 used in Example 32, and the same evaluation as in Example 32 was performed thereto. The results are shown in Table 3.

### Comparative Examples 7-8

Devices were produced by following the same procedure as in Example 32 with the exception that Comparative Compound 2 and 3 were used in place of Exemplified Compound No. 1 used in Example 32, and the same evaluation as in Example 32 was performed thereto. The results are shown in Table 3.

TABLE 3

| | | | | Durability | | |
|---|---|---|---|---|---|---|
| | | Initial | | | | Luminance |
| | | Applied | | Current | Initial | after 100 |
| | Exemplary | Voltage | Luminance | Density | Luminance | hours |
| | Compound No. | (V) | (cd/m²) | (mA/cm²) | (cd/m²) | (cd/m²) |
| Ex. 32 | 1 | 5 | 1500 | 20.0 | 580 | 500 |
| Ex. 33 | 2 | 5 | 1200 | 20.0 | 520 | 400 |
| Ex. 34 | 5 | 5 | 1800 | 20.0 | 610 | 510 |
| Ex. 35 | 8 | 5 | 1700 | 20.0 | 570 | 480 |
| Ex. 36 | 9 | 5 | 1400 | 20.0 | 510 | 400 |
| Ex. 37 | 26 | 5 | 1400 | 20.0 | 500 | 420 |
| Ex. 38 | 28 | 5 | 1600 | 20.0 | 530 | 450 |
| Comp. | Comp. Compd. 2 | 5 | 750 | 20.0 | 320 | 180 |
| Ex. 7 | | | | | | |
| Comp. | Comp. Compd. 3 | 5 | 980 | 20.0 | 420 | 120 |
| Ex. 8 | | | | | | |

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2006-025208, filed Feb. 2, 2006, which is hereby incorporated by reference herein in its entirety.

What is claimed is:

1. A fluorene compound represented by the following general formula [III]: 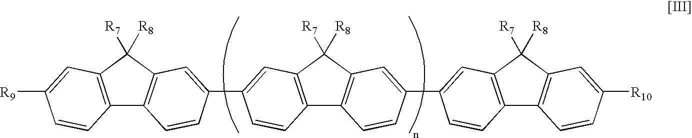
wherein, R₇ and R₈ each represent an alkyl group, a substituted or unsubstituted aralkyl group, a substituted or unsubstituted aryl group, or a trifluoromethyl group with the proviso that at least one of R₇'s and R₈'s is a trifluoromethyl group, R₇'s and R₈'s attached to different fluorene groups may be the same or different from each other, and R₇'s and R₈'s attached to the same fluorene group may be the same or different from each other;
R₉ and R₁₀ each represent a hydrogen atom, a substituted or unsubstituted alkyl group, a substituted or unsubstituted aralkyl group, a substituted or unsubstituted aryl group, a substituted or unsubstituted heterocyclic group, a substituted or unsubstituted fused polycyclic aromatic group, a substituted or unsubstituted fused polycyclic heterocyclic group, a substituted or unsubstituted aryloxy group, a halogen atom, or a cyano group, R₉ and R₁₀ may be the same or different from each other; and
n represents an integer of 1 to 10.

2. An organic light-emitting device comprising a pair of electrodes including an anode and a cathode, and an organic compound layer provided between the pair of electrodes, wherein the organic compound layer comprises the fluorene compound set forth in claim 1.

3. The organic light-emitting device according to claim 2, wherein the organic compound layer is a light-emitting layer.

4. A fluorene compound represented by the following general formula [IV]: 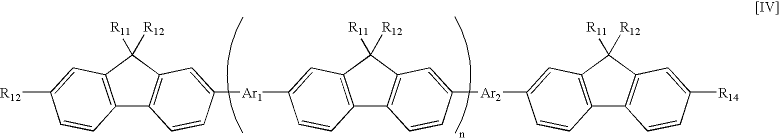
wherein, R₁₁ and R₁₂ each represent an alkyl group, a substituted or unsubstituted aralkyl group, a substituted or unsubstituted aryl group, or a trifluoromethyl group with the proviso that at least one of R₁₁'s and R₁₂'s is a trifluoromethyl group, R₁₁'s and R₁₂'s attached to different fluorene groups may be the same or different from each other, and R₁₁'s and R₁₂'s attached to the same fluorene group may be the same or different from each other;
R₁₃ and R₁₄ each represent a hydrogen atom, a substituted or unsubstituted alkyl group, a substituted or unsubstituted aralkyl group, a substituted or unsubstituted aryl group, a substituted or unsubstituted heterocyclic group, a substituted or unsubstituted fused polycyclic aromatic group, a substituted or unsubstituted fused polycyclic heterocyclic group, a substituted or unsubstituted aryloxy group, a halogen atom, or a cyano group, and R₁₁ and R₁₂ may be the same or different from each other;
Ar₁ and Ar₂ each represent a substituted or unsubstituted divalent aryl group or a substituted or unsubstituted divalent fused polycyclic aromatic group, and Ar₁ and Ar₂ may be the same or different from each other; and
n represents an integer of 1 to 10.

5. An organic light-emitting device comprising a pair of electrodes including an anode and a cathode, and an organic compound layer provided between the pair of electrodes, wherein the organic compound layer comprises the fluorene compound set forth in claim 4.

6. The organic light-emitting device according to claim 5, wherein the organic compound layer is a light-emitting layer.
